(19) United States
(12) Patent Application Publication
(10) Pub. No.: US 2025/0176992 A1
(43) Pub. Date: Jun. 5, 2025

(54) MAGNETIC MILLI-SPINNER FOR UNTETHERED ROBOTIC ENDOVASCULAR SURGERY AND METHODS FOR USE

(71) Applicant: The Board of Trustees of the Leland Stanford Junior University, Stanford, CA (US)

(72) Inventors: Renee Zhao, Stanford, CA (US); Shuai Wu, Stanford, CA (US)

(21) Appl. No.: 18/939,349

(22) Filed: Nov. 6, 2024

Related U.S. Application Data
(63) Continuation of application No. PCT/US2023/021386, filed on May 8, 2023.
(60) Provisional application No. 63/453,152, filed on Mar. 19, 2023.
(60) Provisional application No. 63/339,504, filed on May 8, 2022.

(51) Int. Cl. A61B 17/3207; A61B 17/00; A61B 34/00; A61B 90/00

(52) U.S. Cl. CPC A61B 17/320758; A61B 34/73; A61B 90/37; A61B 2017/00243; A61B 2034/731; A61B 2090/376

(57) ABSTRACT

Devices are provided for performing endovascular surgical procedures. In one example, the device includes a magnetically actuated untethered rotation device, i.e., a magnetic spinner, that can navigate in blood vessels through its spinning-enabled propulsion. The magnetic spinner is capable of providing suction, shear force, carrying load for functions including navigation in multi-branched blood vessels, e.g., for targeted drug delivery, treating brain aneurysm, mechanical thrombectomy, rotablation for plaque removal, and the like.

### RELATED APPLICATION DATA

The present application is a continuation of co-pending International Application No. PCT/US2023/021386, filed May 8, 2023, which claims the benefit of priority to U.S. provisional application Ser. No. 63/453,152, filed Mar. 19, 2023, and U.S. provisional application Ser. No. 63/339,504, filed May 8, 2022, the entire disclosures of which are expressly incorporated by reference herein.

### STATEMENT REGARDING FEDERALLY SPONSORED RESEARCH AND DEVELOPMENT

This invention was made with Government support under Contract 2145601 awarded by the National Science Foundation. The Government has certain rights in the invention.

### TECHNICAL FIELD

The present application relates to medical devices and, more particularly, to devices for performing endovascular surgical procedures, such as thrombectomy, rotablation, drug delivery, treating brain aneurysms, and to systems and methods for using such devices.

### BACKGROUND

The state-of-the-art minimally invasive endovascular technologies rely on a well-trained interventional radiologist to use catheters and guidewires to treat patients for diseases and operations such as stroke, deep vein thrombosis, endovascular coiling or stenting, transcatheter aortic valve replacement, etc. FIG. 1 depicts several examples of cardiovascular diseases frequently treated using these minimally invasive procedures, including an example of a hemorrhagic stroke 10, an example of an ischemic stroke 12, an example of a pulmonary embolism 14, and an example of coronary artery disease 16. These procedures often require navigation of significant arterial tortuosity in elderly patients, which contributes to procedure time and technical failure. In addition, patients often require transfer to hospitals where interventional surgeons are available to perform these procedures.

Percutaneous thrombectomy is a minimally invasive interventional treatment, during which the surgeon inserts a catheter into the patient's blood vessel to remove the blood clot and restore blood flow to the affected area. There are two commonly used mechanical thrombectomy technologies to remove large clots: (i) aspiration thrombectomy, where a continuous vacuum aspiration is induced through a guide catheter to suction out the clot; (ii) stent retriever thrombectomy, where a mesh tube is used pull out the clot. However, the use of stent retrievers and aspiration devices is always associated with a risk of thrombus fragmentation, during which the big clot may break into small pieces (100 μm˜1000 μm) and travel downstream in the blood vessel, potentially blocking the blood flow at multiple other locations and/or leading to new life-threatening emboli that require emergent open surgeries.

Another condition often treated by minimally invasive interventional treatment is atherosclerosis. Atherosclerosis is a condition in which a blood vessel is narrowed due to the build-up of plaque and/or blood clots on the interior wall of the blood vessel. FIG. 2 shows an example of atherosclerosis of a diseased artery 22 of a patient's heart 18, and one example of how it may form. As shown in FIG. 2, plaque 23 forms 24 on the interior wall of the artery 20 and the plaque grows 26. The plaque ruptures 28 and a blood clot forms 30, thereby causing stenosis (narrowing) of the artery.

There are a number of previously disclosed devices and methods for treating atherosclerosis. One procedure for treating atherosclerosis, called angioplasty, uses a balloon catheter and mostly with a stent in the diseased vessel at the location of the stenosis. The balloon and stent widen the narrowed vessel to open up the vessel to blood flow. The stent is typically implanted using one or more intravascular catheters, introducers and guidewires to advance the stent through a patient's vascular system and to place the implant the stent at the stenosed location within the blood vessel. However, stents have various known complications, including stent migration, re-stenosis caused by clots and plaque re-forming around the stent (re-stenosis), among others.

Another procedure to treat atherosclerosis is to remove the plaque and/or clot from the vessel. As an example, FIG. 3 illustrates an intravascular, rotablation device 32 comprising an ablation tool 34 mounted on a drive wire 36. The rotablation device 32 is advanced to the location of the plaque and/or clot, and then the drive wire 36 spins the ablation device 34 to ablate plaque and/or clot. Ablative treatment can create embolisms caused by particles of the plaque and/or clot breaking off, entering the blood flow, and then blocking blood flow. This can result in stroke or damage to other body tissue and organs.

Catheter delivery in tortuous blood vessels is a significant challenge in previously available devices and methods. Accordingly improved devices and methods capable of improved delivery to a target location and, improved devices and methods for performing thrombectomy and/or other endovascular procedures would be useful.

### SUMMARY

The present application is directed to medical devices and, more particularly to devices for performing endovascular surgical procedures, such as thrombectomy, rotablation, drug delivery, treating brain aneurysms, and to systems and methods for using such devices. In one example, the device includes a magnetically actuated untethered rotation device, i.e., a magnetic spinner, that can navigate in blood vessels through its spinning-enabled propulsion. The magnetic spinner is capable of providing suction and shear force, navigating in multi-branched blood vessels, and carrying loads for functions such as targeted drug delivery, treating brain aneurysm, mechanical thrombectomy, rotablation for plaque removal, and the like.

The present disclosure is directed to innovative biomedical devices and methods for performing minimally invasive medical diagnoses and/or treatment. The devices combine an ablation tool and suction capability in a single instrument which allows the integrated functionality of ablating body tissue, and/or capturing and transporting material. Some embodiments disclosed herein are millimeter-scale and self-contained magnetic devices which can be remotely controlled to provide locomotion to navigate within body pathways, spinning to perform ablation, suction and clot removal, and/or actuation to dispense materials within the body. As used herein, the term “self-contained” means that a device is not physically connected to any source of power, or mechanical link for providing motion or actuation. In some cases, the devices also have the capability to dispense substances, such as fluid medicaments. The magnetic spinner devices include one or more magnets that are driven by an external magnetic field driver. By controlling the magnetic field, the devices can be manipulated to perform locomotion, spinning, and/or actuation.

Accordingly, one embodiment disclosed herein is directed to magnetic spinner device having a tubular body sized for introduction into a body lumen of a subject. The tubular body comprises first and second ends and a central lumen extending axially along a central axis between the first and second ends. One or more fins are disposed on an exterior surface of the tubular body. A magnet or a magnet array is also disposed on the tubular body and is configured to cause the tubular body to spin when a magnetic field is applied to the magnetic spinner device. The magnet may be permanent magnets, electromagnets, magnetic composites or other suitable magnets. With varied magnetic field magnitude and frequency combinations, the magnetic spinner can achieve controllable spinning both around the long axis (the axial direction of the tubular body, also referred to as a “central axis”) and the short axis (the central radial direction of the tubular body).

In one aspect of the magnetic spinner device, the one or more fins comprise a plurality of fins extending axially, circumferentially, or helically around the exterior surface of the tubular body at least partially between the first and second ends. The one or more fins may have a constant-size or varying-size cross-section with suitable shapes, such as triangular, square, etc. In still another aspect, the device may also have one or more passages communicating between the exterior surface and the central lumen. The one or more passages may be positioned between adjacent fins communicating between the exterior surface and the central lumen. The one or more passages may be substantially straight or helical slits between the adjacent fins.

In another aspect of the magnetic spinner device, the magnet array may include one magnet or a plurality of magnets spaced apart circumferentially from one another on each of the first and second ends. In yet another aspect, each of the magnets may have the same polarity relative to the central axis such that the magnets generate a rotational force in the same direction around the central axis when the magnetic field is generated.

In still another aspect, the magnetic spinner device may also have a drug delivery member disposed within the central lumen carrying one or more agents. For instance, the drug delivery member may be one or more liquid/solid agents sealed in the central lumen. In another aspect, the drug delivery member may be configured to dissolve in order to release the one or more agents.

In another aspect of the magnetic spinner device, the device may be sized at a millimeter scale in order to travel through small anatomical passages of a patient, such as a cardiovascular system of a patient. For instance, the tubular body may be sized to have an outer diameter between about one and six millimeters (1.0-6.0 mm), and a length between the first and second ends between about 0.9 and ten millimeters (0.9-10 mm).

Another embodiment disclosed herein is directed to a system for performing a minimally invasive medical procedure, such as surgery or diagnosis, using the magnetic spinner device. One such system includes the magnetic spinner device and a magnetic field driver for generating the magnetic field to control rotation of the device. In another aspect, the magnetic field driver includes one or more coils, or one or more magnets, or a combination thereof. In yet another aspect, the magnetic field driver may include a 3-axis Helmholtz coil.

In still another feature, the magnetic field driver may include a controller for controlling the magnetic field magnitude, frequency, and orientation. The controller may be configured to control the magnetic field to control one or more of a speed of rotation of the device, to cause the device to translate within a body lumen, to cause the device to change direction within a body lumen, and to control the spinning around the long axis/short axis of the magnetic spinner.

Another embodiment disclosed herein is directed to a method for performing a minimally invasive biomedical procedure such as a surgery or medical diagnosis, on a subject, using the magnetic spinner devices and systems disclosed herein. One such method includes introducing any of the magnetic spinner device disclosed herein into a body lumen of the subject. Then, a magnetic field is applied to the magnetic spinner device to cause the tubular body to spin about the long axis thereby propelling the device to a target location, or to spin about the short axis. A biomedical procedure is then performed at the target location using the device, for example, spinning about the long axis for gradually releasing a loaded drug member or spinning about the short axis for rapid releasing of a loaded drug delivery member.

In another aspect of the method, the biomedical procedure is an ablation procedure performed by spinning the device while contacting the device against body tissue at the target location to remove the body tissue. In another aspect, the biomedical procedure includes positioning the device proximate an object, and spinning the device using the magnetic field to create a suction which pulls the object toward the device.

The method may further include moving the device by spinning the device using the magnetic field to retract the object out of the body.

In still another aspect, the biomedical procedure may include dissolving a clot within the target location. In yet another aspect, the biomedical procedure may include treating an aneurysm at the target location.

Another embodiment disclosed herein is directed to a millimeter-scale and self-contained, biomedical magnetic spinning device. The spinning device comprises a tubular tool head comprising a tubular body and one or more magnets disposed on the tubular body. The tubular body has a lumen extending axially along the central axis of the tubular body. In one aspect, a magnet may be disposed on one end of the tubular body and the magnet has a hole which aligns with the lumen of the tubular body. The tubular body may have any suitable cross-sectional shape, such as circular (i.e., a cylinder), polygonal (e.g., a square, rectangle, pentagon, hexagon, etc.), etc.

The magnetic spinning device is controllable by applying a magnetic field to the magnet(s) of the device to provide locomotion (i.e., translation), rotation, and/or actuation of the device. In one aspect, the magnetic field is generated and controlled by a magnetic field driver. The magnetic field driver may comprise a 3-axis Helmholtz coil or other suitable magnetic field driver. The magnetic field driver may include a controller for controlling the magnetic field. The controller may be computer-controlled, manually controlled or a combination of computer-controlled and manually controlled.

The magnetic field driver is controllable to dynamically control the magnitude, frequency and orientation of the magnetic field. For example, by continuously rotating the magnetic field, the device's magnet follows the magnetic field, leading to continuous rotation of the device. Spinning of the device about the long central axis of the device can create propulsion in a fluid to cause translation of the device. The device may be steered as it translates by adjusting the plane of rotation of the magnetic field (which is orthogonal to an axis of rotation of the magnetic field). The magnet on the device will follow the plane of rotation, thereby changing the orientation of the device which steers the device. Spinning of the device about the long central axis of the device can also be used to ablate body tissue in contact with the spinning device. The rotation about the long axial direction can also induce the rolling of the magnetic spinner for the translation of the device along a solid material or surface. In addition, spinning of the device about the short central radial axis can cause flipping of the device on the solid material or surface. The device can also jump off a surface by applying an instant magnetic field. This allows the device to overcome larger obstacles that cannot be easily navigated by flipping and/or rolling.

In another aspect, the tubular body of the magnetic spinning device may comprise an origami geometrical configuration comprising surfaces having folding/unfolding capability. In one embodiment, the origami geometrical configuration may comprise triangulated tilted panels forming the tubular body. The triangulated tilted panels are folded to form propeller-like panels which provide propulsion similar to a propeller when the device is spinning.

In another aspect, the origami geometrical configuration is configured to provide a pumping function by the folding/unfolding of the origami geometry. In one embodiment, a first magnet is disposed on a first end of the device with the first magnet having magnetization in a first orientation. A second magnet is disposed on a second end of the device with the second magnet having a magnetization oriented at a second orientation different from the first orientation. The first magnet and second magnet may be permanent magnets, electromagnets, magnetic composites or other suitable magnets. When a magnetic field is applied to the device which causes opposite magnetic torques on the first magnet and second magnet causing them to rotate about the central axis in opposite directions, the device body folds/unfolds (depending on the orientation of the magnetization). The folding/unfolding causes the tubular body to contract/expand in a pumping mechanism. In one aspect, the pumping mechanism can provide controlled release of a substance (e.g., a liquid medicament).

In still another aspect, tubular body of the device may have one or more blades (also referred to as “fins”) disposed on, and extending radially outward from, the exterior surface of the tubular body. The term “blade” does not necessarily denote that the structure has a sharp or cutting edge, but more generally means a blade-like structure which may or may not have a sharp or cutting edge, unless explicitly described as one or the other. The blades may be the same or similar to propeller blades. The blades may increase the propulsion of the device when spinning the device, and may also enhance the ablative function of the tool head.

In yet another aspect, the tubular body of the device has one or more holes through the wall of the tubular body. The holes in the tubular body improve the suction capability of the device. The holes may be slits, apertures, or other through holes in the tubular body. In the case that the device has blades, the holes may be between the blades.

In another aspect, the device is sized to navigate through a body's vascular system. For example, the device has a diameter of from 1 millimeter (mm) to 6 mm and a length of from 0.9 mm to 10 mm. In another aspect, the device may have a diameter of less than 2 mm and a length of less than 3 mm. In other aspects, the device may have a diameter of less than 1 mm and a length of less than 0.9 mm.

Another embodiment disclosed herein is directed to a method of using the magnetic spinning device. The device is introduced into a patient's body via a small incision. Optionally, an introducer is inserted into the incision and the device is inserted into the body through the introducer. The device is navigated to a target location within the body. The device is advanced through a pathway within the body, including body lumens (e.g., blood vessels) and/or body cavities (e.g., cavities within body organs) by applying a magnetic field to the device causing the device to spin, roll, flip and/or jump. For example, the magnetic field may be modulated to continuously rotate the magnetic field causing the device to spin about its long central axis thereby propelling the device within a body fluid, such as blood. The device is steered by adjusting the magnetic field, such as by altering the plane of rotation of the magnetic field. Once the device is advanced to the target location, the device is used to perform a biomedical procedure, such as a diagnostic or treatment procedure. After performing the biomedical procedure, the device is retracted from the body by navigating the device using the magnetic field, same or similar to advancing the device to the target location, except in the opposite direction.

In another aspect of the method, the biomedical procedure is an ablation procedure performed by spinning the device while bearing against body tissue at the target location to remove the body tissue. The device may be translated, and its orientation adjusted, to position the device to ablate the body tissue using the magnetic field. In still another aspect, the body tissue is an occlusion, such as plaque and/or clotting, within a blood vessel.

In another aspect, the method may include using the device to capture and remove an object (e.g., material such as a blood clot or tissue ablated by the device) at the target location. The device is positioned proximate the object and then the device is spun using the magnetic field. The spinning device creates a suction (a low pressure zone) within the lumen of the tubular body which pulls the material toward the device. The object may be pulled into the lumen of the tubular body, or pulled close to the device. The device is then retracted from body by spinning the device using the magnetic field, same or similar to advancing the device to the target location, except that the device is spun in the opposite direction, or the device is turned in the opposite direction. The device is then navigated back out of the body via the pathway. The spinning device pulls the object along with it to capture and remove the object from the body.

In yet another aspect, the method may also include the device dispensing a therapeutic fluid at the target location within the body. As described above, the device may be configured to provide a pumping function, such as by folding/unfolding of an origami geometry. The magnetic field is applied to cause the tubular body to contract, such as by folding the tubular body, to controllably pump a therapeutic fluid, such as a liquid medicament, to the target location. In the embodiment have a first magnet and second magnet on opposite ends of the device, a magnetic field is applied to the device which causes opposite magnetic torques on the first magnet and second magnet causing them to rotate about the central axis in opposite directions. This causes the device body to fold/unfold (depending on the orientation of the magnetization of the first and second magnets) which contracts/expands the tubular body in a pumping mechanism. The pumping mechanism provides controlled release of a substance (e.g., a liquid medicament). Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 shows examples of various endovascular diseases.

FIG. 2 illustrates an example of atherosclerosis of a diseased artery, and how the atherosclerosis may form.

FIG. 3 illustrates a prior art rotablation device on a guidewire ablating a stenosis formed in a blood vessel.

FIGS. 4A-4C illustrate a magnetic spinner device, according to one embodiment disclosed herein.

FIG. 5A illustrates a magnetic spinner device, according to another embodiment disclosed herein.

FIG. 5B illustrates a magnetic spinner device, according to another embodiment disclosed herein.

FIG. 6 illustrates a magnetic spinner device, according to another embodiment disclosed herein.

FIG. 7 shows a comparison of the swimming speed of the magnetic spinner device of FIG. 6 compared to the magnetic spinner device of FIGS. 4A-4C.

FIG. 8A shows a computational fluid dynamics (CFD) simulation of streamline and normalized centerline pressure comparing the streamlines and normalized pressures of the magnetic spinner device of FIG. 6 compared to the magnetic spinner device of FIGS. 4A-4C.

FIG. 8B shows a comparison of particle image velocimetry (PIV) results of the magnetic spinner device of FIG. 6 compared to the magnetic spinner device of FIGS. 4A-4C.

FIG. 9A is a graph of the swimming speed of the spinner device of FIGS. 4A-4C at varying frequencies.

FIG. 9B is a graph showing the maximum rotating frequencies and the maximum speed of the magnetic spinner device of FIGS. 4A-4C under different magnetic field magnitudes.

FIG. 9C is a graphic showing the magnetic spinner device of FIGS. 4A-4C swimming upstream in a pulsatile flow with a peak velocity of 30 cm/s under a rotating magnetic field of B=20 mT, f=100 Hz.

FIG. 9D is a graph showing the quantitative results of displacement and swimming speed of the spinner device 100 upstream swimming as shown in FIG. 9C.

FIG. 10 illustrates front view (left) and a top view (right) of a magnetic field driver and controller for magnetically actuating the magnetic spinner device disclosed herein, according to one embodiment disclosed herein.

FIG. 11 illustrates that the magnetic field generated by the magnetic field driver and controller of FIG. 10 can apply and control a magnetic field to the magnetic spinner device to move and navigate the spinner device 100 through multiple body lumens, and also illustrates that the navigation of the magnetic spinner device 100 may be monitored using external imaging, such as X-ray imaging.

FIGS. 12A-12E illustrate a magnetic spinner device being used to remove the plaque from a blood vessel.

FIGS. 13A-13B illustrate a magnetic spinner device configured to be rotatable around two axes by a magnetic field driver 200, according to another embodiment disclosed herein.

FIG. 14 show a contour plot of the varied combinations of magnetic field magnitude and frequency applied to a magnetic spinner to produce various motion states including a spinning state (triangular dots), a flipping state (square dots), or an unstable state (round dots).

FIG. 15 illustrates a magnetic spinner device configured to carry and release a therapeutic, according to another embodiment disclosed herein.

FIG. 16A shows the rapid drug release rate for the magnetic spinner device of FIG. 15 upon a flipping motion of the magnetic spinning device.

FIG. 16B shows a slower drug release rate for the magnetic spinner device of FIG. 15 upon a flipping motion of the magnetic spinner device.

FIG. 17 illustrates the magnetic spinner device of FIG. 15 to treat an aneurysm, according to one embodiment disclosed herein.

FIGS. 18A-18G illustrate a magnetic spinner system 300 in combination with the magnetic spinner devices disclosed herein, according to another embodiment disclosed herein.

FIG. 19 illustrates a magnetic spinner device having a body formed of foldable panels, according to still another embodiment disclosed herein.

FIGS. 20A-20B illustrate a magnetic spinner device similar to that of FIG. 19 configured to provide a pumping function by the folding/unfolding of the origami geometry.

FIG. 21 shows a blood clot and a deep vein thrombosis blocking blood flow through a vein.

FIGS. 22A-22G illustrate a method of using the magnetic spinner devices disclosed herein to create suction to capture and move an object.

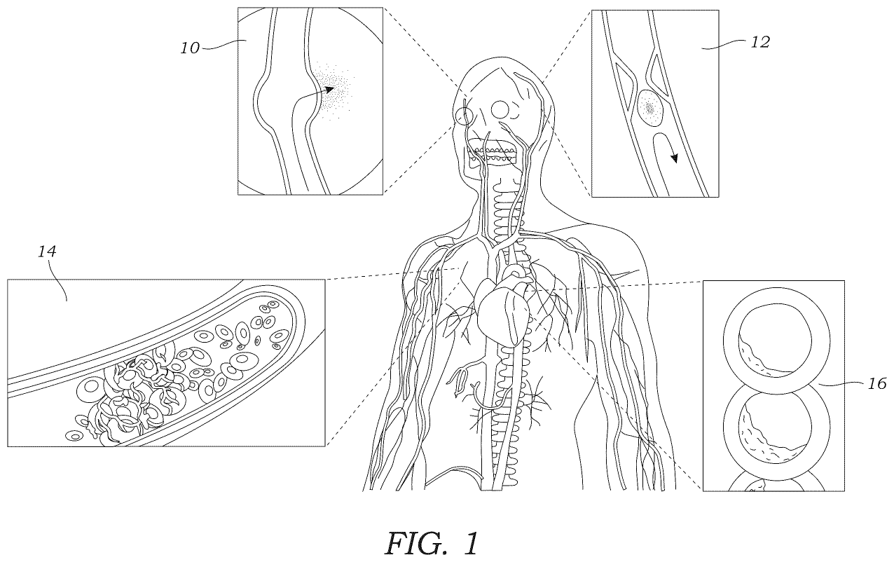

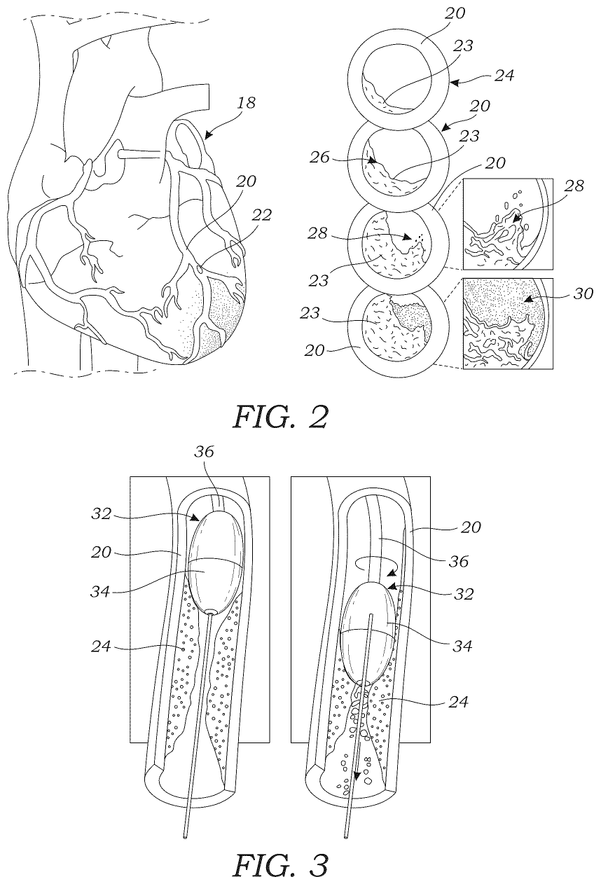

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

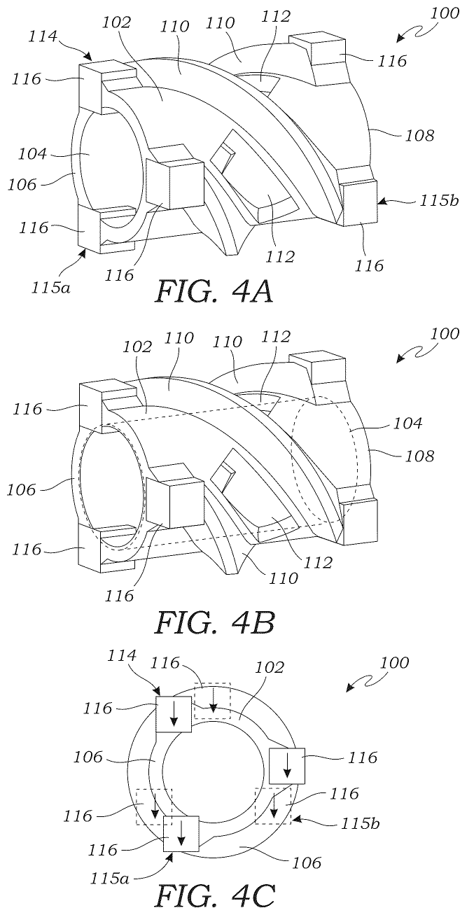

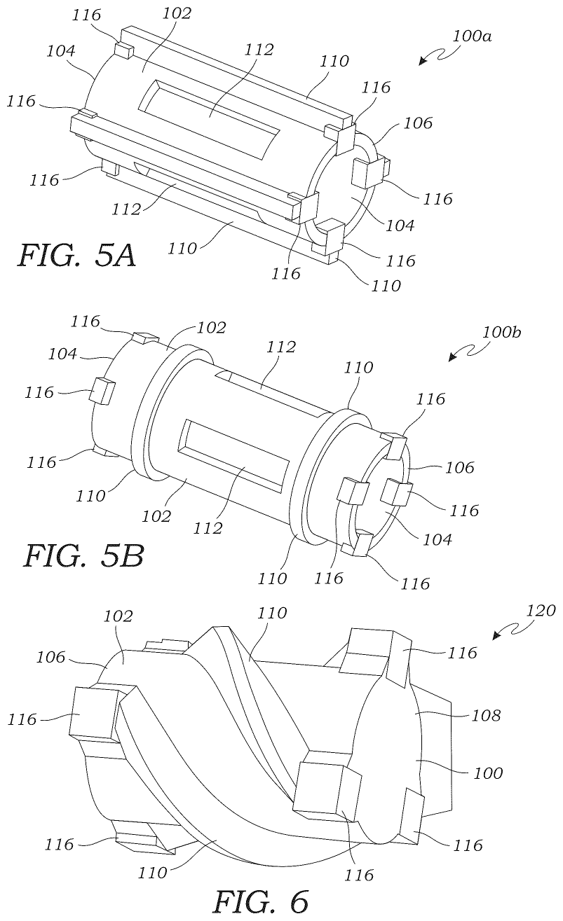

Turning to the drawings, FIGS. 4A-4C show one embodiment of a magnetic spinner device 100 that generally includes a cylindrical tubular body 102 including a central lumen 104 extending between a first end 106 and a second end 108 thereof. The magnetic spinner 100 also has a plurality of helical fins 110 on its exterior surface. In between each pair of two helical fins 110, a cut, slit, or other passage 112 may be provided through the wall of the tubular body 102 that extends from the outer surface to the central lumen 104, i.e., that connects the inside of the spinner 100 with the outside. Instead of helical fins 110, a magnetic spinner device 100a may have fins 110 which alternatively extend axially as shown in FIG. 5A, or a magnetic spinner device 100b may have fins 110 which alternatively extend circumferentially as shown in FIG. 5B, on the exterior surface of the tubular body 102 at least partially between the first and second ends 106, 108.

As best seen in FIG. 4C, the spinner device 100 includes an array 114 of magnets 116, e.g., including two sets of magnets 115a, 115b on the first and second ends. As shown, each set may include three magnets 116 on each side, e.g., spaced apart circumferentially (i.e., angularly spaced) from one another on each of the first and second ends 106, 108. The number of magnets 116 may vary based on the size of the spinner device 100 and other parameters of the device 100, such as magnetic field strength of the magnets 116, desired swimming speed, ablation rotation speed, applied magnetic field strength etc. The magnetization direction (i.e., the polarity orientation) of the magnets 116 may be the same and in the same plane of a cross-section (perpendicular to the long axis the tubular body 102) of the spinner device 100, e.g., such that each of the magnets 116 has the same polarity relative to a central axis of the tubular body so that the magnets 116 generate a rotational force in the same direction around the central axis when a magnetic field is applied to the spinner device 100.

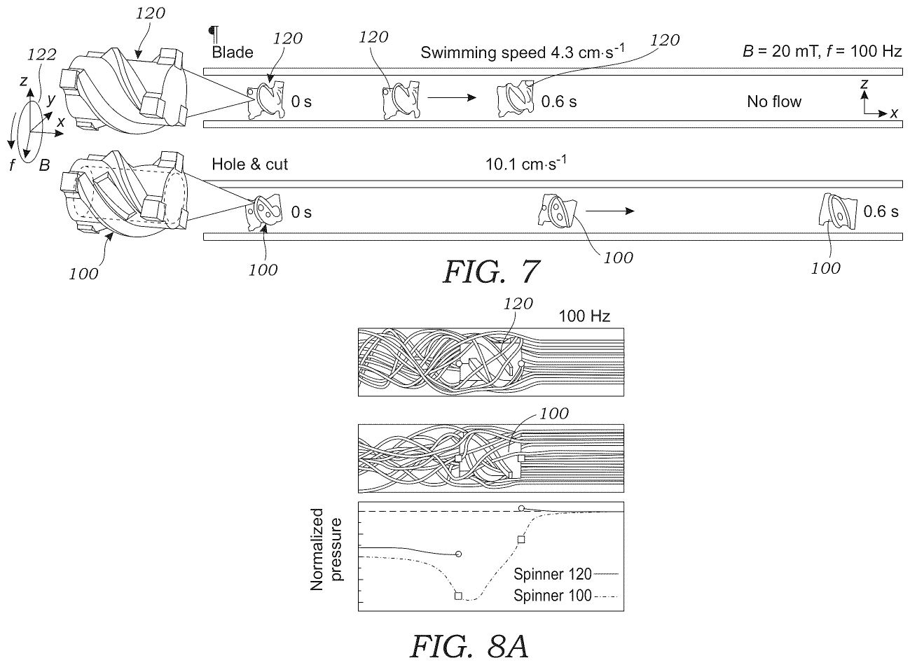

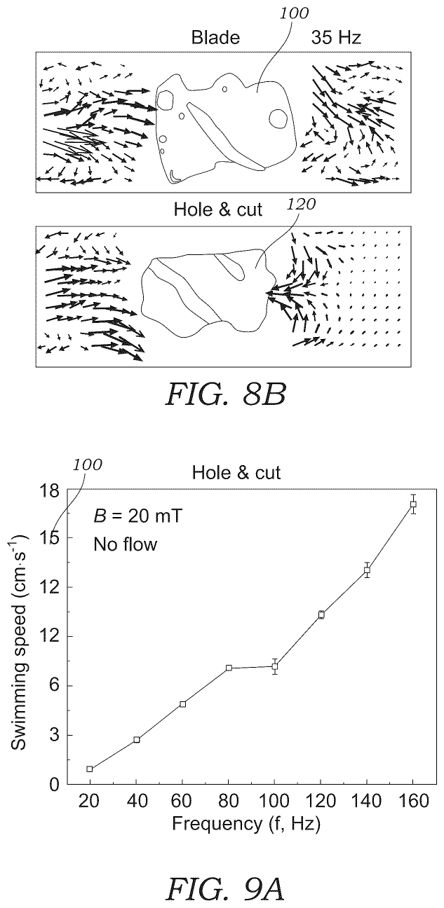

The central lumen 104 may 1) allow blood shunting, meaning that blood can flow through the central lumen 104 within a blood vessel to minimize obstruction of normal blood flow; and/or 2) may improve the translation or “swimming” speed of the device 100 through a body lumen, e.g., through a suction mechanism, compared to a spinner device 120 without a central lumen (and also without passages 112 as there is no central lumen 104), e.g., as shown in FIG. 6. FIG. 7 shows a comparison of the translation or “swimming” speed of the respective spinner devices 100 and 120 when subjected to a rotating magnetic field having a magnitude B=20 mT and a frequency f of 100 Hz. FIG. 7 shows that including a central lumen 104 and passages 112 on the spinner device 100 increases the swimming speed of the spinner device 100. FIGS. 8A-8B compare results of the spinner device 100 to the spinner device 120 under various operating conditions. FIG. 8A shows a computational fluid dynamics (CFD) simulation of streamline and normalized centerline pressure comparing the streamlines and normalized pressures of the spinner device 120 not having a central lumen and passages and the spinner device 100 having central lumen 104 and passages 112, both spinning at 100 Hz. The simulation in FIG. 8A shows an external flow from right to left is imposed with the speed obtained as shown in FIG. 7.

FIG. 8B shows a comparison of particle image velocimetry (PIV) results of the magnetic spinner device of FIG. 6 compared to the magnetic spinner device of FIGS. 4A-4C, both spinning at 35 Hz, swimming from left to right in FIG. 8B.

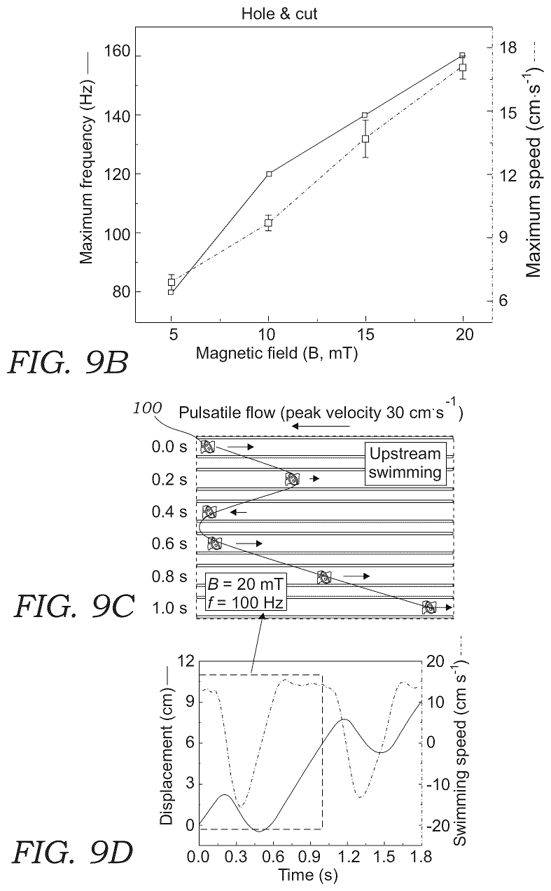

FIG. 9A shows the swimming speed of the spinner device 100 at varying frequencies and FIG. 9B shows the maximum rotating frequencies of the and maximum speed of the spinner device 100 under different magnetic field magnitudes. FIG. 9C is a graphic showing the magnetic spinner device 100 swimming upstream in a pulsatile flow with a peak velocity of 30 cm/s under a rotating magnetic field of B=20 mT, f=100 Hz. FIG. 9D is a graph showing the quantitative results of displacement and swimming speed of the spinner device 100 upstream swimming as shown in FIG. 9C.

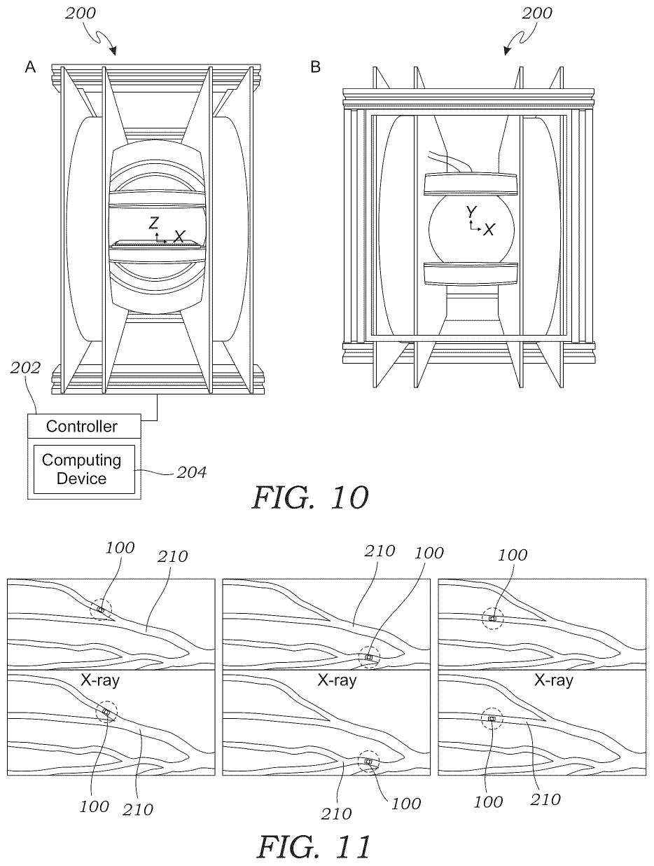

The magnetic spinner device 100 is controllable by applying a magnetic field to the magnetic array 114 of the device 100 to provide locomotion (i.e., translation), rotation, spinning, flipping, and/or actuation of the spinner device 100. The magnetic field is generated by a magnetic field driver 200, such as a 3-axis, 3-dimensional Helmholtz coil drive, as shown in FIG. 10 from two different sides. The magnetic field driver 200 has a controller 202 for dynamically controlling the magnitude, rotational speed, and orientation of the magnetic field applied to the magnetic spinner device 100. The controller 202 may be computer-controlled by a computing device 204, manually controlled or a combination of computer-controlled and manually controlled.

The magnetic field driver 200 can cause the magnetic spinner device 100 to spin continuously rotating the magnetic field, such that the device's magnetic array 114 follows the magnetic field, leading to continuous rotation of the magnetic spinner device 100. Spinning the device 100 about the long axis or short axis of the device 100 in a fluid (e.g., blood, saline, or other bodily fluids) creates propulsion causing translation of the device 100, or other motions such as rolling, flipping, jumping. The magnetic spinner device 100 is steered as it translates by adjusting the plane of rotation of the magnetic field (which is orthogonal to the axis of rotation of the magnetic field). The magnetic array 114 on the robot will follow the plane of rotation, thereby changing the orientation of the device 100 which steers the device 100.

As shown in FIG. 11, the magnetic field may be controlled to facilitate navigation of the spinner device 100 through multiple body lumens, e.g., within multiple branches of blood vessels 210 within the patient's vasculature, such as the pulmonary arteries 210 shown in the top three images. The translation and rotation of the spinner device 100 allow the device 100 to easily navigate in multi-branch blood vessels under control of the magnetic field. Optionally, the navigation of the device 100 may be monitored using external imaging, such as fluoroscopy or other x-ray imaging guidance, as shown in the bottom three images of FIG. 11.

The spinner device 100 can also be moved by rolling or flipping the spinner device 100 while in contact with a solid surface by spinning of the device 100 about the long axial axis or about a short central radial axis perpendicular to the axial axis, respectively, as described in more detail below. The spinner device 100 can also be made to jump off a surface by applying an instant magnetic field, thereby allowing the spinner device 100 to overcome larger obstacles that cannot be easily navigated by flipping and/or rolling.

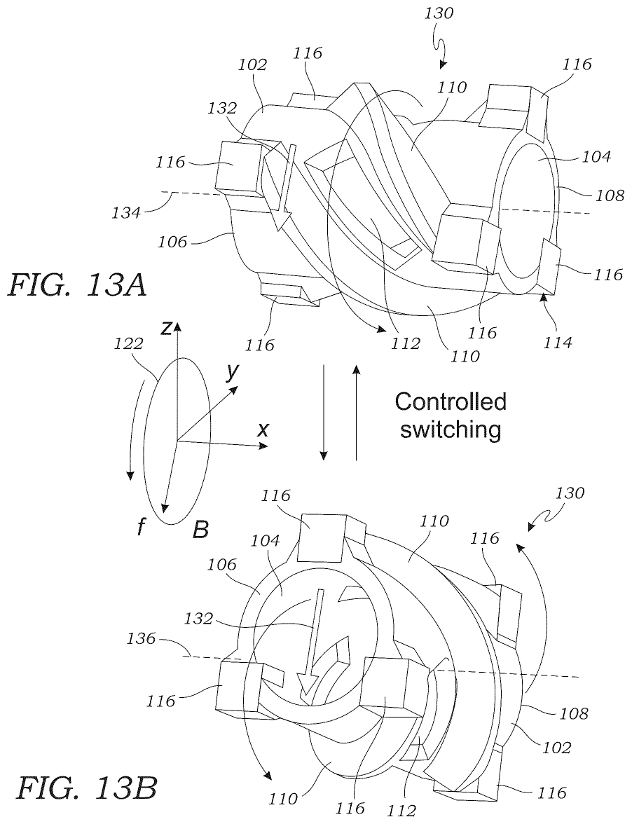

During use, e.g., after introducing the magnetic spinner device 100 into a patient's vasculature and/or other body lumen, e.g., via a catheter, sheath, or other delivery device (not shown), a rotational magnetic field is applied to drive actuation of the spinner device 100, e.g., using the systems and methods as illustrated in FIG. 10, and described herein. The plane of magnetic field rotation determines the swimming direction of the spinner device 100, which is perpendicular to the rotation plane, as shown by the graphic 122 in FIG. 7. For example, the configuration shown in FIGS. 4 and 7 shows the spinner device following the right-hand rule (see graphic 122 in FIG. 7 showing the plane of rotation at a frequency f and swimming direction along the x-axis) to determine the swimming direction, meaning the curl direction of the four fingers is the spinning direction of the spinner device 100, and the thumb direction is the swimming direction. The magnetic field may be programmed to change the spinning mode of the spinner device 100 for other functions such as liquid drug delivery and solid object delivery, e.g., as described with respect to the embodiments in FIGS. 13A-17.

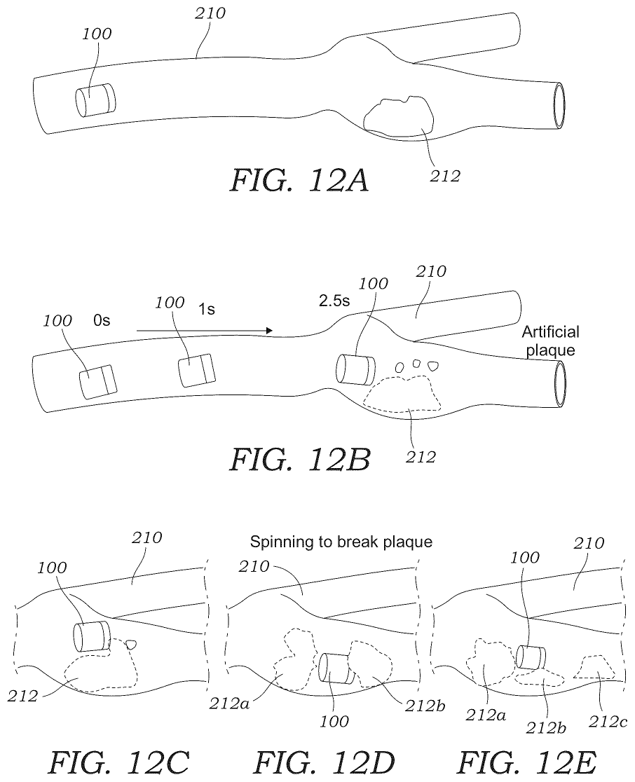

The spinner device 100 can also be used for mechanical thrombectomy, rotablation, and the like. FIGS. 12A-12E illustrate ablating body tissue by spinning of the device 100 about the central axis of the spinner device 100 while the device 100 is in contact with body tissue. In FIG. 12A, the spinner device 100 is shown in a model of a blood vessel 210 proximal to a stenosis 212 in the blood vessel 210 formed by plaque and/or a blood clot. As illustrated in FIG. 12B, the spinner device 100 is spun by applying a magnetic field using the magnetic field driver 200 to propel (i.e., move) and navigate the spinner device 100 distally along the blood vessel 210 to the target location of the stenosis 212 by modulating the orientation and/or rotational speed of the magnetic field. As shown in FIG. 12C, the spinner device 100 is moved into contact with the stenosis 212 and continuously spun while contacting the stenosis 212 such that the blades ablate the stenosis. FIG. 12D shows that the spinner device 100 has ablated the stenosis 212 to break it up into multiple pieces 212a and 212 b. FIG. 12E shows the spinner device 100 continuing to be spun in contact with the stenosis 212 to further break up the plaque into multiple pieces 212a, 212b and 212c, until all of the desired plaque material is removed. As shown in FIGS. 12A-12E, the spinner device 100, may be used for mechanical thrombectomy, rotablation, and the like, similar to the devices described in the application incorporated by reference herein. For example, once positioned adjacent a clot, the spinner device may be rotated to provide suction to effectively capture the clot. Optionally, the spinning motion may generate a relatively large shear force to mechanically dissolve the clot, e.g., by squeezing red blood cells from the clot to significantly reduce the clot volume.

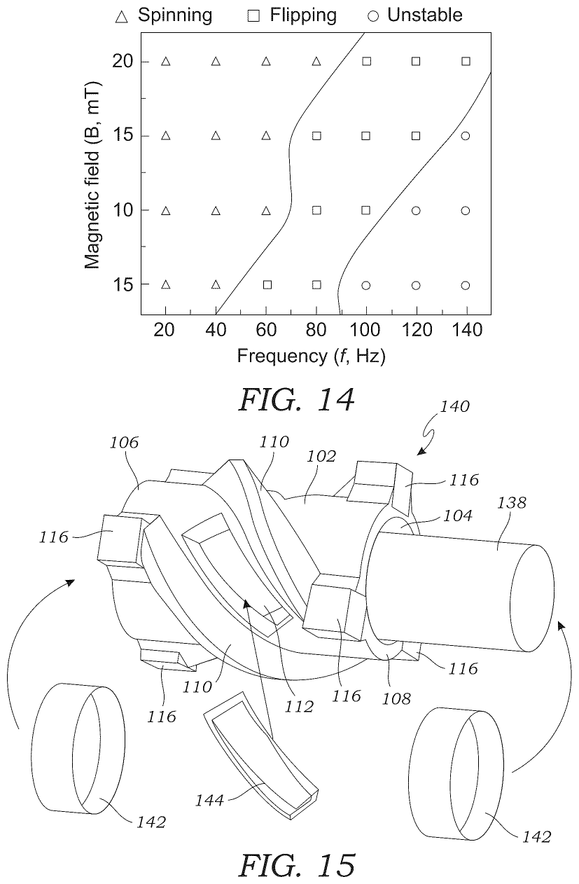

Turning to FIGS. 13A-13B, another embodiment of a magnetic spinner device 130 configured to be rotatable around two axes by the magnetic field driver 200 is illustrated. The magnetic spinner device 130 is the same as the magnetic spinner 100, except that the magnetic array 114 has a net magnetization in a radial direction as shown by the net magnetization arrow 132. Accordingly, the spinner device 130 is capable of being rotated around two axes, the long axis (central axis) through the tubular body 102, as shown in FIG. 13A, and the short axis along the central radial direction, as shown in FIG. 13B. When applying a constantly rotating magnetic field in the yz-plane as indicated by the coordinate system graphic 122, the spinner 130 can achieve two types of motions: spinning when the magnetic field rotational axis 134 is along the spinner long axial direction, and flipping when the magnetic field rotational axis 136 is perpendicular to the spinner long axial direction. The spinning and flipping of the spinning device 130 are switchable by controlling the combination of the magnetic field magnitude B and frequency f. For example, as shown in the contour plot in FIG. 14, under varied combinations of B from 5 to 20 mT, with a step of 5 mT, and f from 20 to 140 Hz, with a step of 20 Hz, the spinner motion states of a spinning state (triangular dots), a flipping state (square dots), or an unstable state (round dots) in a 4 mm diameter channel. The different spinner motion states are separated by the dashed lines. As shown in FIG. 14, for a given magnetic field magnitude B, the spinner device 130 is inclined to accomplish spinning motion at low f, and then changes to flipping motion with an increasing f, until being unstable when f is too high for the spinner device 130 to follow. By applying a larger B, the spinner device 130 achieves a higher f spinning, namely, realizing faster swimming until switching to a flipping motion as the f is further increased.

The magnetic spinner devices 100 and 130 may also be configured to carry and release a therapeutic 138, such as a drug 146 or occlusive material 148, as shown in the embodiment of FIG. 15. The magnetic spinner device 140 is loaded with a drug 138 in the central lumen 104. For example, the therapeutic 138 may be in a powder, gel or liquid form. In order to contain the therapeutic 138 in the central lumen 104, the spinner device 140 has end seals 142 on each the first and second ends 106, 108, and passage seals 144 on each of the passages 112. The end seals 142 and passage seals 144 may be soluble covers which dissolve to open the first and second ends 106, 108 and passages 112. Alternatively, the end seals 142 and passage seals 144 may be any other suitable sealing device which can seal the first and second ends 16, 108 and passages 112, and selectively open to allow the therapeutic 138 to be released from the spinner device 140. The therapeutic 138 is released by the spinning device 140 by opening the end seals 142 and passage seals 144 (for example, dissolving the soluble covers), and then applying a magnetic field to spin and/or flip the spinning device 140 which causes the therapeutic to be released out from the central lumen 104 through the first and second ends 106, 108 and the passages 112.

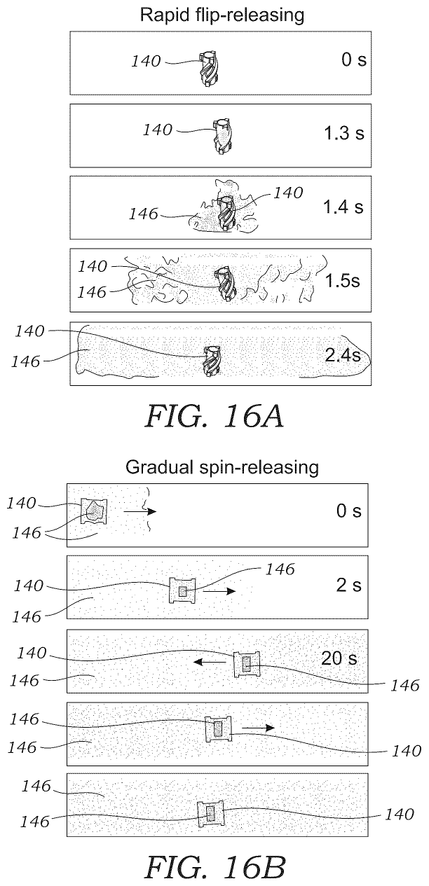

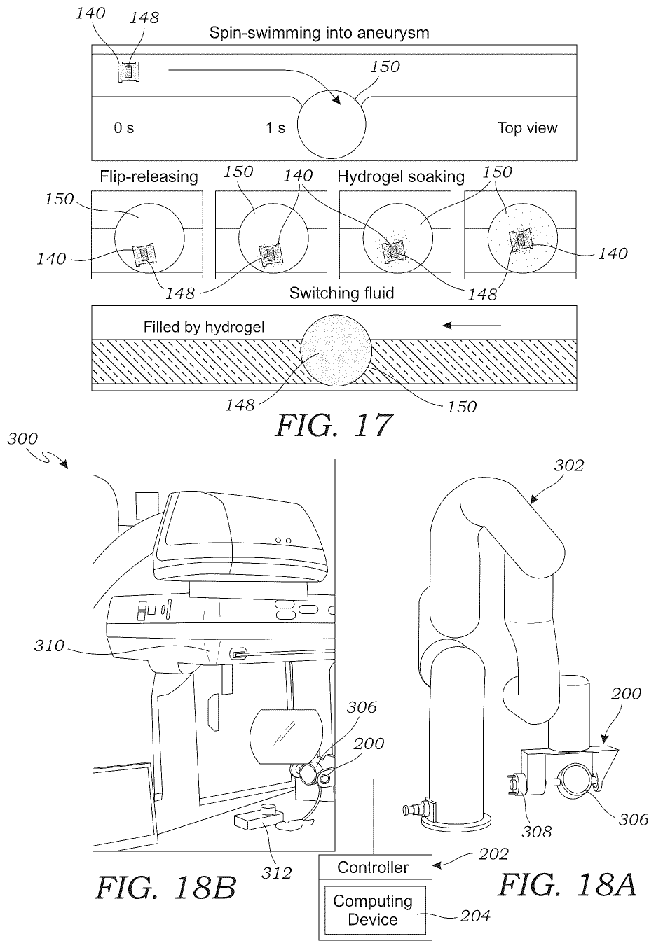

The ability to control the switching between spinning and flipping motions of the magnetic spinner device 140 also enables the spinner device 140 to provide different drug release mechanisms and corresponding drug release, as illustrated in FIGS. 16A-16B. FIGS. 16A-16B show test results for utilizing the magnetic spinning device 140 for drug delivery using a powdered material to simulate a powdered drug 146. FIG. 16A shows the rapid drug release rate for a flipping motion of the magnetic spinning device 140. As shown in FIG. 16A, once the magnetic spinning device 140 reaches the targeted position and the covers 142, 144 are dissolved, a magnetic field for flipping motion is applied causing the device 140 to flip such that the drug 146 is rapidly released mostly through the first and second ends 106, 108 (and may also release to a smaller extent through the passages 112). FIG. 16A shows that significant therapeutic is released in just 1.5 seconds, and then continues to diffuse to a wide area of the vessel within an additional one second. In comparison, FIG. 16B shows the much slower drug release rate for a spinning motion of the spinner device 140. As shown in FIG. 16B, when a magnetic field for spinning motion is applied to the spinner device 140, the drug 146 is released mostly through the passages 112 (and also through the first and second ends 106, 108) at a much more gradual rate measured in tens of seconds, and the drug 146 again diffuses to a wide area. Also, as shown in FIG. 16B, because the spinning motion of the spinner device 140 causes the spinner device 140 to move, the direction of the spinning motion is alternated to keep the spinner device in the targeted position. In addition, the speed/frequency of the spinning or flipping of the spinner device 140 may be controlled by controlling the applied magnetic field to control the delivery rate of the drug 146, such as to set the delivery rate to a desired delivery rate. The higher the speed/frequency of the spinning or flipping of the spinner device 140, the higher the delivery rate of the drug 146. Turning to FIG. 17, the spinner device 140 configured for releasing a therapeutic 138 may be used to treat an aneurysm 150, e.g., a cerebral aneurysm 150 within a patient's brain. A brain aneurysm 150 is a sac-like structure that grows on the cerebral artery wall and potentially ruptures, leading to bleeding into the brain and hemorrhagic stroke. Current treatments aim to prevent further flow into the aneurysm, either by clipping the aneurysm base or by filling the aneurysm with metal coils. However, the former method requires open brain surgery and the latter can be problematic for aneurysms at hard-to-reach regions. The magnetic spinner device 140 with a loaded therapeutic provides another option for aneurysm treatment. As shown in FIG. 17, the central lumen 104 of the spinner device 140 is loaded with an occlusive material 148, such as expandable material which expands in volume when exposed to liquid. For example, the occlusive material 148 may be a suitable hydrogel material. The spinner device 140 is advanced by swimming through the blood vessels into the aneurysm 150 by the spinning motion. Once within the aneurysm 150, the covers 142, 144 are dissolved, and the spinner device 140 is put into a flipping and/or spinning motion by the applied magnetic field to release the loaded occlusive material 148. The released occlusive material 148 expands within the aneurysm 150 to occupy substantially the whole aneurysm 150. As shown in FIG. 17, the expanded occlusive material 148 prevents further blood flow into the aneurysm 150.

Any of the magnetic spinner device 100, 120, 140 disclosed herein may also be effectively used in combination with medical imaging systems and robotic systems, to provide a precisely controllable magnetic spinner system 300. As described herein, the motion of the spinner devices 100, 120, 140 is controllable by dynamically controlling the magnitude, orientation and/or magnitude of the magnetic field applied by a magnetic field driver 200, and that the magnetic field driver 200 may be controlled by a controller 202 which can be computer-controlled. Accordingly, the spinner devices 100, 120, 140 can realize precise and more complex motion beyond straight-line locomotion. Moreover, the precisely controllable complex motion does not require extra actuation components on the spinner devices 100, 120, 140, but only a programmable magnetic field control system.

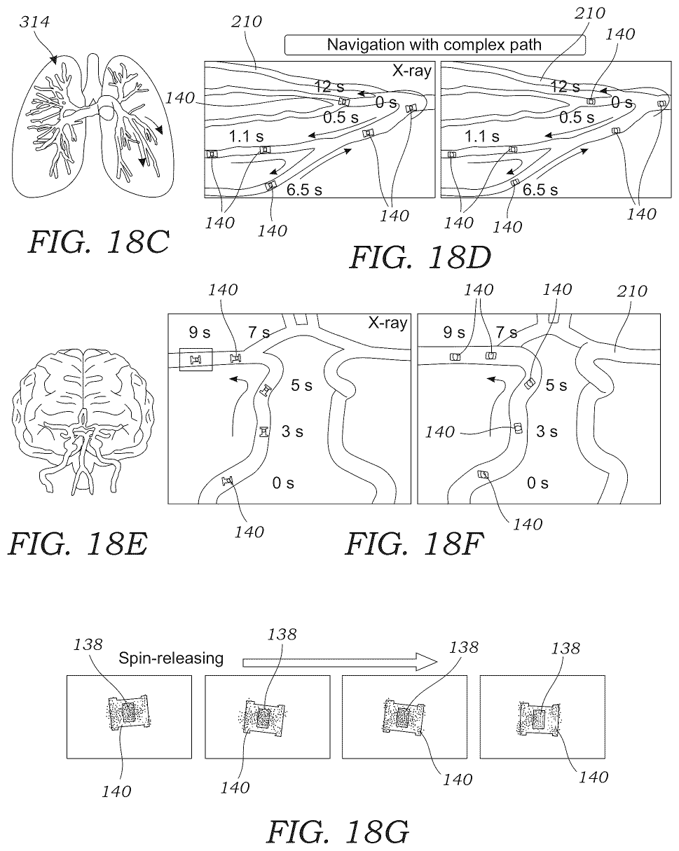

To that end, FIGS. 18A-18G illustrate magnetic spinner system 300 in combination with the magnetic spinner device 140. It is understood that any of the magnetic spinner devices, including spinner devices 100, 120 may also be used in the system 300. FIGS. 18A-18B illustrate a magnetic spinner system 300 having a computer-controlled robotic arm 302 which carries a magnetic field driver 200. The robotic arm 302 is a multiple-degree-of-freedom robot arm, such as a 4-axis, 5-axis, or 6-axis robot. The magnetic field driver 304 functions as the end-effector of the robotic arm 302. The magnetic field driver 200 is shown FIG. 18A as a rotating magnet 306 driven by a motor 308. Alternatively, the magnetic field driver 200 may be a 3-axis Helmholtz coil or other suitable magnetic field driver mounted as the end-effector of the robotic arm 302. In the case of the rotating magnet 306 driven by a motor 308, the magnetic field magnitude is controlled by the relative distance between the rotating magnet 306 and the spinner device 140 which is controlled by the robotic arm 302, the spinning frequency is controlled by the motor 308, and the magnetic field orientation is simply programmed by the direction of the magnet's rotational axis as controlled by the robotic arm 302.

The magnetic spinner system 300 also includes a medical imaging system 310, which may be any suitable medical imaging system, such as X-ray, ultrasound, fluoroscopy, etc. Because of the high density of magnets 116 of the spinner device 140 (typically at least 7600 kg m⁻³), X-ray imaging technology works well to provide real-time guidance of the navigation and drug release of the spinner device 140, as the spinner device 140 is highly visible even when obstructed by a skull bone (1600 to 1900 kg m⁻³). Moreover, X-ray imaging has the merits of relatively low cost, case of usage, and high-quality imaging. Thus, in the example of FIGS. 18A-18B, the medical imaging system 310 is an X-ray imaging system 310.

As shown in FIG. 18B, for experimental purposes, flow models are put in between the X-ray source and X-ray detector of the X-ray imaging system 310 while the rotating motor on the robotic arm 302 is arranged to the sides of the flow model 312 with a programmed trajectory. The navigation capability with a complex path is first demonstrated in a real-size pulmonary artery model 314, as shown in FIG. 18C. With controlled external magnetic field magnitude and frequency, the magnetic spinner device can spin and swim in multiple artery branches with different sizes (˜1.5 mm to 10 mm), as illustrated in FIG. 18D. From the X-ray imaging, the location of the spinner device 140 is evident by the front and back magnets 116 denoted by the dark dots in FIG. 18D. Guided by the X-ray imaging, the spinner device 140 can controllably travel into one or the other branch at a branching point as desired by programming the robotic arm 302 and magnetic field driver 200 to control the orientation of the rotating magnetic field (e.g., controlling the rotational axis of the magnet 306). As illustrated in FIG. 18D, the spinner device 140 first reaches the middle branches in 1.1 seconds. Due to structural symmetry, the spinner device 140 can be retrieved following the same path by simply reversing the rotating direction of the applied magnetic field, without requiring a 180-degree turn in such a confined space. Then, the spinner device 140 is guided to reach the bottom branch in 6.5 s and eventually reaches the top branch in 12 s.

Turning to FIGS. 18F-18G, the multifunctional magnetic spinner device's 140 targeted drug release capability is also demonstrated in a much more tortuous real-size cerebral artery model shown in FIG. 18E. As the therapeutic 138, dense powders are loaded and sealed in the central lumen 104 of the spinner device 140 to demonstrate the X-ray guided targeted drug release process. In this case, the central portion of the spinner device 140 is also visible because of the high density of the therapeutic material 138, as illustrated in FIG. 18F. Utilizing a computer-programmed navigation path, the spinner device is shown to swim to the targeted middle cerebral artery in 9 s, after which the covers 142, 144 are dissolved. The spinning device 140 is then spun or flipped to release the therapeutic 138. In the example of FIGS. 18F-18G, the spinning device 140 is spun, as described above, to slowly and gradually release the therapeutic 138 from the central lumen 104. The therapeutic release process can be visualized on the X-ray system 310 as the progressive absence of central dark color on the spinner device 140 under X-ray imaging, and only the magnets 116 are visible eventually when the loaded dense therapeutic has been fully released, as shown in the FIG. 18G. Similar to the pulmonary artery example of FIGS. 18C-18D, the spinner device 140 is retrievable by swimming back after releasing the therapeutic 138.

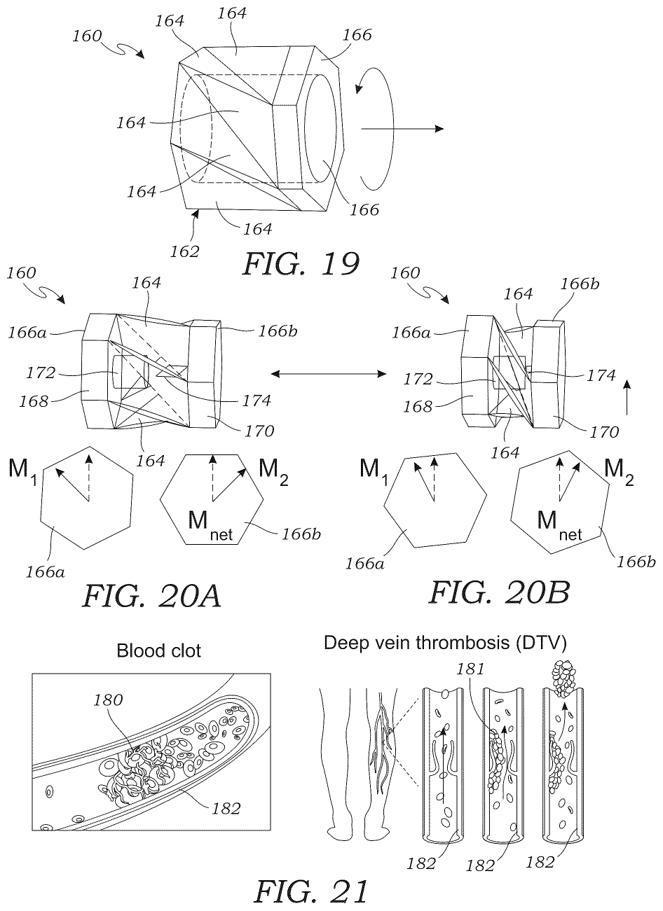

Referring now to FIG. 19, another embodiment of a magnetic spinner device 160 is shown. The magnetic spinner device 160 is similar to the magnetic spinner device 100, 120 and 140, except that instead of a cylindrical body with fins, it has a tubular body 162 having a polygonal cross-section formed by tilted panels 164 forming the exterior of the tubular body 162. In the illustrated embodiment, the magnetic spinner device 160 has a tubular body 162 having a hexagonal cross-sectional shape, but the tubular body 162 may have any suitable cross-sectional shape, such as circular (i.e., a cylinder), polygonal (e.g., a square, rectangle, pentagon, hexagon, etc.), etc. The tubular body 162 has a central lumen 166 extending axially along the central axis of the tubular body 162.

The spinner device also has one or more magnets 166. In the illustrated embodiment, the spinner device 160 has a single magnet 166 disposed on one end of the tubular body 162 and having a hole which aligns with the central lumen 164 of the tubular body 162. The spinner device 160 may have additional magnets 166, such as a magnet 166 on the other end of the tubular body 162, or a magnetic array 114 comprising a plurality of magnets 116, as described herein.

The spinner device 160 is controllable in the same manner described herein for spinner devices 100, 120 and 140, to provide locomotion (i.e., translation), rotation, and/or actuation of the robot. As with the spinner devices 100, 120 and 140, the magnetic field driver can cause the spinner device 160 to spin continuously about the central axis of the spinning device robot in a fluid (e.g., blood, saline, or other bodily fluids) to create propulsion thereby producing a swimming action. The spinning device 160 can be steered as it translates by adjusting the plane of rotation of the magnetic field (which is orthogonal to an axis of rotation of the magnetic field). The magnet 166 of the spinning device 160 will follow the plane of rotation, thereby changing the orientation of the spinning device 160 which provides steering of the spinning device 160 as it moves.

Similar to the functions of the spinning devices 100, 120 and 160, described herein, the spinning device 160 can be used to ablate body tissue by spinning the device 160 robot about the central axis of the device 160 while the device 160 is in contact with body tissue. The spinning device 160 can also be moved by rolling or flipping the device 160 while in contact with a solid surface by flipping the device 160 about an axis lateral (e.g., perpendicular) to the axial axis. The spinning device 160 can also be made to jump off a surface by applying an instant magnetic field, thereby allowing the spinning device 160 to overcome larger obstacles that cannot be easily navigated by flipping and/or rolling.

In another aspect, the tubular body 162 has an origami geometrical configuration comprising surfaces having folding/unfolding capability to allow the tubular body 162 to be axially compressed, such as by magnetic force. The origami geometrical configuration is formed from triangulated tilted panels 164 which form a propeller-like structure and function like propellers to create propulsion when the spinning device 160 spins about its central axis.

As shown in FIGS. 20A-20B, the magnetic spinning device 160 may be configured to provide a pumping function by the folding/unfolding of the origami geometry. The magnetic spinner device 160 has a first magnet 166a disposed on a first end 168 of the tubular body 162 which has a magnetic field in a first orientation M1. The spinner device 160 also has a second magnet 166b disposed on the second end 170 of the tubular body 162 which has a magnetic field oriented at a second orientation M2 different from the first orientation M1. The spinner device 160 also has a container 172 (which may hold dye, medicament, a therapeutic 138 or other fluid) and a puncture device 174 (e.g., a needle) directed at the container, inside the central lumen 164. As depicted in FIG. 20B, when a magnetic field B is applied to the spinner device 160, it causes the first magnet 166a and second magnet 166b to generate opposite magnetic torques causing the magnets 166a, 166b to rotate about the central axis in opposite directions, thereby causing the origami tubular body to fold or unfold, depending on the orientation of the magnetic field. As shown in FIG. 20B, the folding causes the tubular body 162 to contract which causes the puncture device 174 to puncture the container 172, thereby releasing the contents of the container 172. The contraction of the tubular body 162 also pushes into the container 172 and reduces the interior volume of the tubular body 162 in a pumping mechanism which pumps the contents of the container out of the spinner device 160. With this pumping functionality, the spinner device 160 can be used to dispense a therapeutic 138 at a target location, similar to the process described herein for the spinner device 140. Thus, the spinner device 160 may be propelled and navigated to a target location using the magnetic field to spin and steer the device 160 to the target location. The magnetic field is then applied to cause the tubular body 162 to contract to puncture the container 172 and controllably pump the therapeutic 138 fluid to the target location.

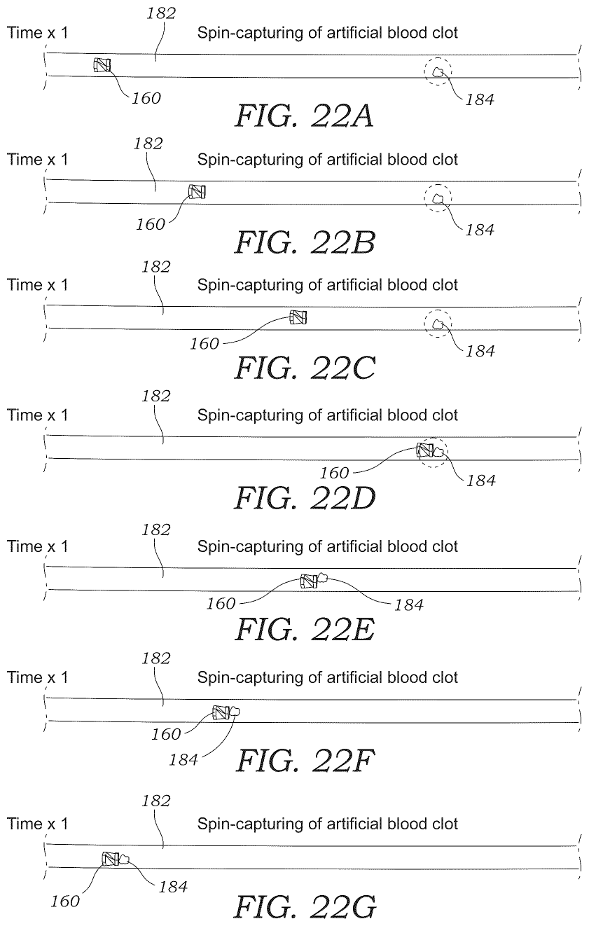

The spinner devices 100, 120, 140 and 160 can also create suction to capture and remove objects and material from a body lumen or body cavity. For example, FIG. 21 illustrates a blood clot 180 and a deep vein thrombosis 181 (a blot clot in a deep vein of the leg) blocking blood flow through a vein 182. FIGS. 22A-22G illustrate the use of the spinner device 160 to create suction to capture and remove an object 184 (such as a thrombosis (blood clot 180), ablated material, etc.). It is understood that any of the spinner devices 100, 120 and 140 disclosed herein may be used similarly. The spinning device 160 is sized to navigate through a body's vascular system, such as arteries, veins and/or other body lumens and body cavities. The spinning device may have a diameter of from about 1 mm to about 6 mm and a length of from about 0.9 mm to about 10 mm. Optionally, the robot may have a diameter of less than 3 mm, and a length of less than 3 mm.

As shown in FIGS. 22A-22D, the spinner device 160 is spun about its central axis in a first direction by applying a magnetic field using the magnetic field driver 200 to propel the spinner device distally into proximity with an object 184 (e.g., a simulated blood clot) at a target location within a simulated blood vessel. At the same time, the spinner device 160 is steered by changing the orientation of the magnetic field. Once at the target location, as shown in FIG. 22E, the spinner device 160 is spun in a second direction opposite to the first direction to create a suction to pull the object 184 toward the robot and to propel the spinner device 160 in the proximal direction. As the spinner device 160 moves proximally, as shown in FIGS. 22E-22G, the suction pulls the object 184 such that the object 184 follows the spinner device 160. In some cases, the object 184 may be pulled into the central lumen 164 of the tubular body 162, although the object 184 shown in the example of FIGS. 22A-22G is larger than the diameter of the central lumen 164. The spinner device 160 may be retracted through the blood vessels all the way out of the body to remove the object 184 from the body.

The methods of using the magnetic spinner devices 100, 120, 140 and 160 to perform an endovascular procedure may also include the steps for introducing the device through a guide tube to a release location, for instance, within the vascular system of a subject's body. For example, the method may include inserting an introducing sheath through an incision of the subject's body. Then, a guide tube may be inserted through the introducing sheath and navigated through the vascular system of the subject's body to position a distal opening of the guide tube at a device release position. For example, the guide tube may be any suitable tube such as a shuttle sheath, a catheter, a guide catheter, or the like. The magnetic spinner device is then inserted into the guide tube. A pusher device is inserted into the guide tube proximal of the spinner device and the pusher device is advanced within the guide tube to push the spinner device through the guide tube until the device reaches the distal opening of the guide tube. Then, the spinner device is released out through the distal opening of the guide tube into the vascular system at the release position. The spinner device is then moved and navigated using a magnetic field to position the spinner device at a target position for performing a biomedical procedure, as described herein. A biomedical procedure at the target location using the spinner device, as disclosed herein. The spinner device is then removed from the vascular system as described above.

It should be further understood that the spinner devices 100, 120, 140 and 160 disclosed herein may be used for any endovascular applications that require delivering liquid or solid objects and/or procedures that involve mechanically break things into pieces or volume reduction of the bio-objects such as rotablation for plaque removal, using the same or similar procedures described herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. A device for performing an endovascular procedure, comprising:
a tubular body sized for introduction into a body lumen of a subject, the tubular body comprising first and second ends and a central lumen extending axially along a long axis (in an axial direction of the tubular body) between the first and second ends;
one or more fins on an exterior surface of the tubular body; and
a magnet or a magnet array on the tubular body configured to cause the tubular body to spin around the long axis (the axial direction of the tubular body) or a short axis (a central radial direction of the tubular body) when a magnetic field is generated around the tubular body.

2. The device of claim 1, wherein the one or more fins comprise a plurality of fins extending axially, circumferentially, or helically around the exterior surface of the tubular body at least partially between the first and second ends.

3. The device of claim 1, further comprising one or more passages communicating between the exterior surface and the central lumen.

4. The device of claim 2, further comprising one or more passages between adjacent fins communicating between the exterior surface and the central lumen.

5. The device of claim 4, wherein the one or more passages comprise substantially straight or helical slits between the adjacent fins.

6. The device of claim 1, wherein the magnet array comprises a plurality of magnets spaced apart circumferentially from one another on each of the first and second ends.

7. The device of claim 6, wherein each of the magnets has the same polarity relative to the long axis such that the magnets generate a rotational force in the same direction around the long axis when the magnetic field is generated.

8. The device of claim 6, wherein a net magnetization of the magnet array is in a direction which allows an applied magnetic field to cause the device to move in a flipping motion about the short axis.

9. The device of claim 1, further comprising a drug delivery member disposed within the central lumen carrying one or more agents.

10. The device of claim 9, wherein the drug delivery member comprises a porous body carrying the one or more agents.

11. The device of claim 9, wherein the drug delivery member is configured to dissolve to release the one or more agents.

12. The device of claim 1, wherein the tubular body has an outer diameter between about one and six millimeters (1.0-6.0 mm), and a length between the first and second ends between about 0.9 and ten millimeters (0.9-10 mm).

13. A system for performing endovascular surgery, comprising:
a device according to claim 1; and
a magnetic field driver for generating the magnetic field to control rotation of the device.

14. The system of claim 13, wherein the magnetic field driver comprises one or more coils, or one or more magnets.

15. The system of claim 13, wherein the magnetic field driver comprises a 3-axis Helmholtz coil.

16. The system of claim 13, wherein the magnetic field driver comprises a controller for controlling the magnetic field.

17. The system of claim 16, wherein the controller is configured for controlling the magnetic field to control one or more of a speed of rotation of the device, to cause the device to translate within a body lumen, and to cause the device to change direction within a body lumen.

18. A method for performing an endovascular procedure within a subject's body, comprising:
introducing a device into a body lumen of the subject, the device comprising a tubular body including a central lumen extending axially along a long axis (in an axial direction of the tubular body) between first and second ends of the tubular body, and one or more fins on an exterior surface of the tubular body;
generating a magnetic field to cause the tubular body to spin about the long axis thereby propelling the device to a target location; and
performing a biomedical procedure at the target location using the device.

19. The method of claim 18, wherein biomedical procedure is an ablation procedure performed by spinning the device while contacting the device against body tissue at the target location to remove the body tissue.

20. A method for performing an endovascular procedure within a subject's body, comprising:
inserting an introducing sheath through an incision of the subject's body;
loading a guide tube through the introducing sheath;
navigating the guide tube in a vascular system of the subject's body to position a distal opening of the guide tube at a device release position;
inserting a device according to claim 1 into the guide tube;
inserting a pusher device into the guide tube and push the device through the guide tube with the pusher device until the device reaches the distal opening of the guide tube;
releasing the device out through the distal opening of the guide tube into the vascular system at the release position;
manipulating the device using a magnetic field to cause navigate to a target position; and
performing a biomedical procedure at the target location using the device.
